(19) United States
(12) Patent Application Publication
(10) Pub. No.: US 2010/0260684 A1
(43) Pub. Date: Oct. 14, 2010

(54) USE OF 3-ALKANOYLOXYMETHOXYCARBONYL NITROXIDE ESTERS AS OXIMETRY PROBES FOR MEASUREMENT OF OXYGEN STATUS IN TISSUES

(76) Inventors: Joseph P.Y. Kao, Silver Spring, MD (US); Gerard M. Rosen, Rockville, MD (US); Ke-Jian Liu, Albuquerque, NM (US)

Correspondence Address: MOORE & VAN ALLEN PLLC, P.O. BOX 13706, Research Triangle Park, NC, 27709, US

(21) Appl. No.: 12/601,853

(22) PCT Filed: Jun. 2, 2008
(86) PCT No.: PCT/US08/65495

§ 371 Date: May 6, 2010

Related U.S. Application Data
(60) Provisional application No. 60/932,443, filed on Jun. 1, 2007.

(51) Int. Cl. A61K 49/06

(52) U.S. Cl. 424/9.33

(57) ABSTRACT

The present invention provides for lipophilic, labile alkanoyloxymethyl esters of nitroxides that cross the blood-brain barrier, and after hydrolysis with esterases therein, the corresponding anionic nitroxides are intracellularly entrapped at levels sufficient to permit O₂ measurements by electron paramagnetic resonance spectroscopy.

### CROSS REFERENCE TO RELATED APPLICATION

The present application claims priority to U.S. Provisional Patent Application No. 60/932,443 filed on Jun. 1, 2007, the contents of which are hereby incorporated by reference herein for all purposes.

### GOVERNMENT RIGHTS

This invention was made with U.S. government support awarded from the following agency: NIH grant P30ES012072. The U.S. has certain rights in this invention.

### BACKGROUND OF THE INVENTION

1. Technical Field

The present invention relates to imaging agents and uses thereof. More particularly, the present invention relates to precursor imaging probes for targeting oxygen in tissues for investigative and diagnostic imaging.

2. Related Art

Molecular oxygen is fundamental to many aspects of brain physiology. For example, in neurons, it is essential for the synthesis and catabolism of neurotransmitters such as dopamine, norepinephrine and serotonin. Importantly, diverse pathophysiologies, including stroke, drug abuse, and neurodegenerative disorders such as Alzheimer's and Parkinson's diseases, are associated with acute or chronic alterations in brain O₂ concentration.

Real-time estimates of O₂ levels in brain tissue in living animals are important criteria in the treatment of many cancers and in understanding the pathology of stroke, epilepsy, and traumatic brain injury. However, measurement of O₂ levels in animal tissues using different imaging modalities is not a trivial task. Previously, O₂ in biological systems has been measured by invasive methods, such as the Clark-type electrodes and fluorescence quenching of a ruthenium dye, and by minimally invasive techniques, including ¹⁹F-NMR spectroscopy and blood oxygen level-dependent (BOLD) imaging.

Notably, although O₂ is paramagnetic, electron paramagnetic resonance (EPR) spectroscopy cannot directly detect this molecule at 37° C. and requires the development of molecular probes that can report O₂ concentrations. Interestingly, since molecular oxygen is paramagnetic, it broadens the electron paramagnetic resonance (EPR) spectral lines of other paramagnetic species, such as nitroxides or trityl radicals. Therefore, measured changes in the EPR spectral line-widths of spin probes have been used to estimate O₂ concentrations in homogenous solutions. With the development of low-frequency EPR spectroscopy and imaging, it is now feasible with the appropriate probe to reliably estimate local O₂ concentrations in vivo, in situ, and in real time. Changes in EPR spectral linewidth for paramagnetic species have been used to measure O₂ concentrations in homogenous solutions (2,4). The concentration of O₂ in the vasculature of tumors has been successfully measured by EPR imaging using trityl radicals (2). However, because of their large size and ionic charge, trityl radicals cannot cross the blood-brain barrier or be intracellularly localized. Therefore, trityl radicals are unsuitable for O₂ measurements in the brain.

Notably, an obstacle to the development of minimally invasive EPR imaging agents for use in measuring O₂ concentrations in the brain is the difficulty of localizing O₂— sensitive probes to specific sites of interest. To overcome this limitation, paramagnetic lithium phthalocyanine (LiPc) particles have been stereotaxically implanted for brain O₂ measurements in living animals at specific sites by EPR spectroscopy. However, implantation of LiPc is an invasive surgical procedure. Importantly, stroke causes spatially heterogeneous changes in brain tissue oxygenation, and LiPc implantation at single or multiple sites can provide only limited information regarding O₂ distribution in different regions of the brain.

Although nitroxides are widely used to study membrane fluidity and the redox status of cells, they have had limited utility in vivo, owing at least in part to their poor biostability. In 1995 it was demonstrated that the anion of 3-carboxy-2,2,5,5-tetramethyl-1-pyrrolidinyloxyl is highly resistant to bioreduction (13) Shen (4) found that 3-acetoxymethoxycarbonyl-2,2,5,5-tetramethyl-1-pyrrolidinyloxyl did cross the blood-brain barrier and accumulated in brain tissue where, after esterase hydrolysis, 3 -carboxy-2,2,5,5-tetramethyl-1-pyrrolidinyloxyl was liberated and entrapped. Notably, it was found from Shen that high concentration of 3-carboxy-2,2,5,5-tetramethyl-1-pyrrolidinyloxyl is required to accurately measure O₂ levels in the brain tissue by electron paramagnetic resonance (EPR) imaging but the concentration of the precursor must be low enough not to alter the brain physiology. However, it was found that high levels of the precursor 3-acetoxymethoxycarbonyl-2,2,5,5-tetramethyl-1-pyrrolidinyloxyl was required to reach the necessary concentration of 3-carboxy-2,2,5,5-tetramethyl-1-pyrrolidinyloxyl in the brain tissue which could alter the brain physiology.

Thus it would be advantageous to provide precursors that easily passes the blood brain barrier, can be delivered to the brain at a concentration that does not affect brain physiology, have biostability and can provide sufficient levels of 3-carboxy-2,2,5,5-tetramethyl-1-pyrrolidinyloxyl for distribution throughout brain tissue for mapping the O₂ concentration therein.

### SUMMARY OF THE INVENTION

The present invention relates to precursor oximetry probes that easily cross the blood brain barrier, are highly resistant to bioreduction and upon hydrolysis with an esterase liberate 3-carboxy-2,2,5,5-tetramethyl-1-pyrrolidinyloxyl, wherein the precursor oximetry probes include at least one compound having the structure:

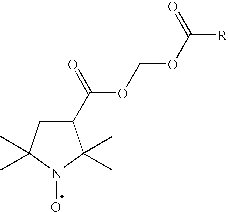

wherein R is a straight-chain alkyl group, represented by the general formula (CH₂)ₙCH₃ wherein n can be 1 to 15.

In another aspect the present invention relates to a method of determining concentration levels of O₂ in brain tissue of a subject in need of such determination, the method comprising:

administering to the subject at least one compound having the structure

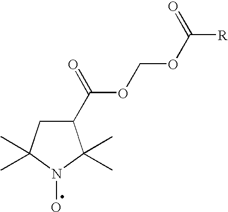

wherein R is a straight-chain alkyl group, represented by the general formula (CH₂)ₙCH₃ wherein n can be 1 to 15, in an amount sufficient to cross the blood-brain barrier and accumulates in brain tissue; and
determining the EPR spectral linewidth of the administered compound in the brain tissue wherein the linewidth increases relative to the concentration of O₂ in the tissue.

In yet another aspect, the present invention relates to a method of determining concentration levels of O₂ in brain tissue in need of such determination, the method comprising:
administering to the vasculature leading to the brain a 3-alkanoyloxymethoxycarbonyl nitroxide ester having the structure

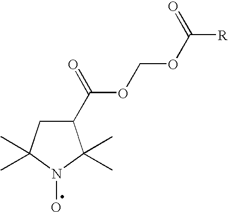

wherein R is a straight-chain alkyl group, represented by the general formula (CH₂)ₙCH₃ wherein n can be 1 to 15, in an amount sufficient to cross the blood-brain barrier and accumulated in brain tissue for hydrolysis therein thereby generating 3-carboxy-2,2,5,5-tetramethyl-1-pyrrolidinyloxyl having the structure:

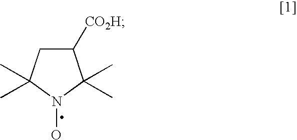

and
determining the amount of accumulated 3-carboxy-2,2,5,5-tetramethyl-1-pyrrolidinyloxyl and/or any remaining 3-alkanoyloxymethoxycarbonyl nitroxide ester to determine the levels of O₂ is such tissue. Preferably, n is 2 to 10, more preferably 2 to 7 and most preferably 2 to 5.

In a still further aspect, the present invention relates to a method for providing an EPR oximetry probe to map O₂ levels in testing tissue, the method comprising:

administering to the tissue a compound having the structure

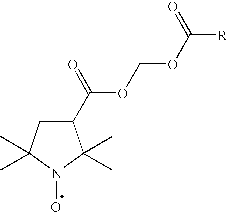

wherein R is a straight-chain alkyl group, represented by the general formula (CH₂)ₙCH₃ wherein n can be 1 to 15.

Another aspect of the invention relates to a method to determine the level of oxygen in tissue, the method comprising:
administering intraperitoneally,intravenously or intraarterially to a subject a compound having a structure

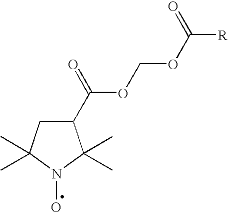

wherein R is a straight-chain alkyl group, represented by the general formula (CH₂)ₙCH₃ wherein n can be 1 to 15, in an amount sufficient to accumulate in the tissue; and
determining the concentration of O₂ in the imaged tissue.

A further aspect of the present invention provides for a method to evaluate oxygen concentration in a biological system, including the steps of (1) introducing physiologically acceptable paramagnetic material to the biological system, (2) applying a magnetic field and/or an electromagnetic field to the biological system, and (3) determining the EPR spectra of the biological system, wherein the paramagnetic material is a compound having a structure

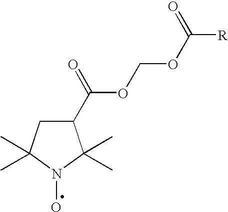

wherein R is a straight-chain alkyl group, represented by the general formula (CH₂)ₙCH₃ wherein n can be 1 to 15.

These and other aspects and advantages of the invention are evident in the description which follows and in the accompanying drawings.

### BRIEF DESCRIPTION OF THE FIGURES

FIG. 1 is a schematic showing the diffusion of the nitroxides of the present invention into a brain cell, where esterase hydrolysis liberates nitroxide [1], which is anionic at physiologic pH. The anionic nitroxide [1] is membrane-impermeant and therefore is retained intracellularly.

FIG. 2 shows synthesis schemes for labile esters [3]-[7].

FIG. 3 shows 3-carboxy-2,2,5,5-tetramethyl-1-pyrrolindinylosyl [1] and optimally effective labile ester derivatives of the present invention [3], [4] and [7].

FIG. 4 shows the results of intracellular loading of nitroxide [1] after Jurkat lymphocytes were incubated with various concentrations of the labile esters [2]-[7].

FIG. 5 show the in vivo EPR mapping of pO2 in the brain of an ischemic mouse.

FIG. 6 shows the increase in EPR spectral line width of nitroxide [1] as a function of the percentage of O2.

FIG. 7 shows measurements of the concentration of nitroxide in the head of a mouse.

FIG. 8 show the percentage of nitroxide [2] and [4] entrapped in the brain at 10 minutes after initial injection as shown in FIG. 7.

FIG. 9 show the percentage of nitroxide [2] and [4] entrapped in the brain at 20 minutes after initial injection as shown in FIG. 7.

FIG. 10 shows a logP vs. logk′ calibration line used to convert the logk′ values of esters 2-7 into the corresponding logP values.

### DETAILED DESCRIPTION OF THE INVENTION

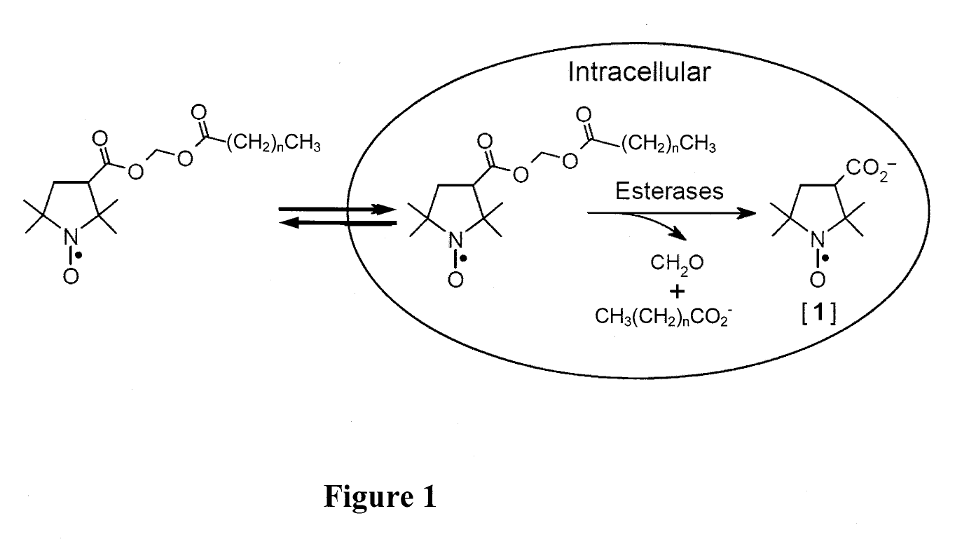

The present invention provides for lipophilic, labile alkanoyloxymethyl esters of nitroxides that can cross the blood-brain barrier, and after hydrolysis, the corresponding anionic nitroxide is intracellularly entrapped at levels sufficient to permit O₂ measurements as shown in FIG. 1. The utility of nitroxides as imaging agents depends critically on their ability to accumulate in tissues at high levels. Further, the disclosed labile esters have been found to deliver carboxylates intracellularly and provide for the delivery of nitroxide imaging agents into relevant tissue for quantitation of O₂ therein.

The nitroxides of the present invention are detectable by electron paramagnetic resonance (EPR) spectroscopy. With the development of advanced imaging instrumentation, images of intact biological tissues and organs are available based on a measurement and detection of the stable free radical of a nitroxide. Pursuant to this invention, nitroxide levels in the body may be maintained for a prolonged period of time allowing both improved image contrast and longer signal persistence. Moreover, unlike certain existing image-enhancing agents, the nitroxides disclosed here are capable of crossing the blood-brain barrier.

Materials and methods are also described herein for the preparation and administration of stable nitroxides in several forms. In particular, inactive, relatively non-toxic precursors or derivatives of membrane-permeable nitroxides are described which are converted in vivo by enzymes described herein to biologically active nitroxides.

A distinct advantage of the nitroxides of the present invention is the capability to deliver the image-enhancing function to several regions of the body, such as the vascular compartment, interstitial space, and intracellular regions or to a particular region based on selective permeability of the biological structure by utilizing known methods of administration which provide targeted or localized effect. It will be appreciated by those skilled in the art, the invention can be particularly applied to the cardiovascular system by intravenous or intraarterial delivery of one or more of the nitroxides described herein.

Notably, the nitroxides of the present invention having a naturally occurring unpaired electron, provide a further advantage, that being, there is essentially no background noise when used in EPR systems. Nitroxides of the present invention can also act as contrast agents to add metabolic information to the morphological data already available from MRI. For example, by substituting various functional groups on the nitroxides, it is possible to manipulate properties including solubility, biodistribution, in vivo stability and tolerance.

In view of the stable chemical nature of the presently described nitroxides, the compounds disclosed here can be administered by various routes. The membrane-permeable nitroxides can be administered parenterally, intraperitoneally, intravenously, intra-arterially, intratumorally, orally or with an implantable device, such as into tumor tissue for the slow and ongoing release of the nitroxides for monitoring purposes. The nitroxides may be delivered with or without a pharmaceutically acceptable carrier in a non-toxic amount. Further, the nitroxides can be administered prior to or during an imaging scan, such as using electron paramagnetic resonance spectroscopy.

Generally, the amount of such nitroxide will depend on the size of the area being monitored and the method of delivery. The amount should be sufficient to interact with the concentration of O₂ typically found in testing tissue and adjusted upward as O₂ concentration is found to increase. Generally, the amount delivered intravenously or intraperitoneally would be in the range from about 0.01 to about 5 mg/g of body weight. If delivered intratumorally, then the mass and weight of the tumor should be considered.

The nitroxides of the present invention provide several advantages including routine measurement of O₂ in a patient's tissue by monitoring the responses of at least one nitroxide of the present invention that is placed in proximity to the testing biological tissue. While there are a large number of medical conditions for which the measurement of the O₂ in tissues is useful, there are several reasons why the present invention is especially beneficial within the two pathologies of peripheral vascular disease and cancer: (1) a large number of patients have these diseases; (2) there is practical clinical value in modifying the treatment of patients afflicted with these diseases on the basis of measurements of oxygen concentration; and (3) there is relative ease in measuring O₂ by using the nitroxides of the present invention.

Notably, peripheral vascular disease of the legs is a frequent problem in the elderly and in patients with diabetes. The clinical care of these patients is difficult because of a lack of an objective method in the prior art to determine the oxygenation of the dermis, hypodermis, and muscles, i.e., the regions at risk for symptoms and/or hypoxic damage resulting from poor circulation. The patient's response to drugs or surgical procedures is also very difficult to determine, when based solely on the reports of the patient, especially relative to long term trends. The invention, however, alleviates these difficulties and enables the physician to obtain objective and routine measurements from several areas, on a repetitive basis, and without discomfort or danger to the patient. It can also monitor the effectiveness of both drug and surgical therapies in a rapid, non-subjective fashion.

The invention provides other advantages in the treatment of cancer, especially by radiation, which is critically dependent on the concentration of oxygen. This has been confirmed recently during clinical treatment of patients utilizing Clark-type microelectrodes in the measurement of O₂. Despite the invasiveness of Clark-type approach, these studies have clearly indicated how valuable it is to have direct measurements of O₂ in tumors. In accord with the use of the nitroxides of the present invention, each patient with a suitable anomaly, e.g., brain and neck tumors, breast cancer, skin cancers, and tumors involving lymph nodes should have an initial evaluation of O₂ to determine whether a conventional treatment is likely to be effective. Thereafter, the determination of O₂ in the tumor anomaly is repeatedly monitored, during therapy, to determine if the treatment is affecting the anomaly as expected. The radiation therapist can utilize this information to suitably alter the treatments in a time frame that is much faster than existing methods: currently, the physician finds out if hypoxic regions are present within the patient only after she learns that the tumor persists after several months.

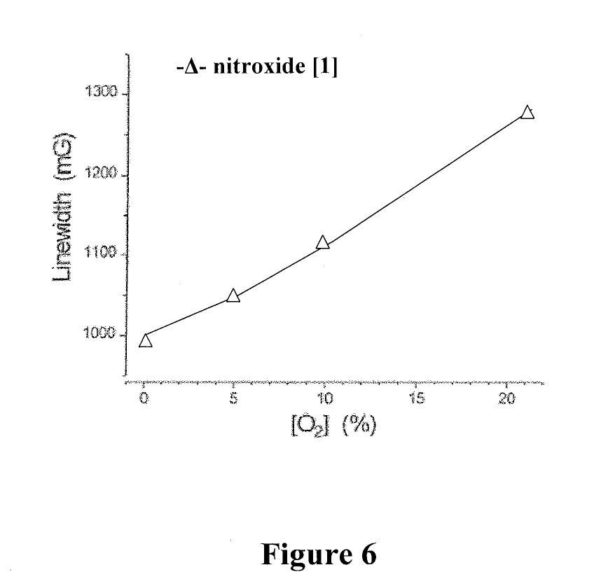

The nitroxides of the present invention having a structure

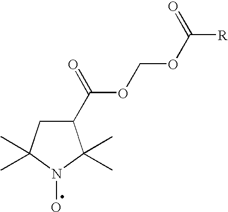

wherein R is a straight-chain alkyl group, represented by the general formula (CH₂)ₙCH₃ wherein n can be 1 to 15, are paramagnetic material. Each will have a EPR signal spectrum with a peak-to-peak line width that is calibrated with known oxygen concentrations to directly determine O₂ concentration in vivo. A set of calibration data is compiled for different concentrations of O₂, including in the absence of oxygen. Notably, as the O₂ content in the testing tissue increases the line-width increases. When nitroxides of the present invention are within biological tissues, the shape of the EPR spectra will be between the previously determined O₂ concentration range values, which is used to determine the in vivo concentration of oxygen, as shown in FIG. 6.

### Examples

Materials and Methods.

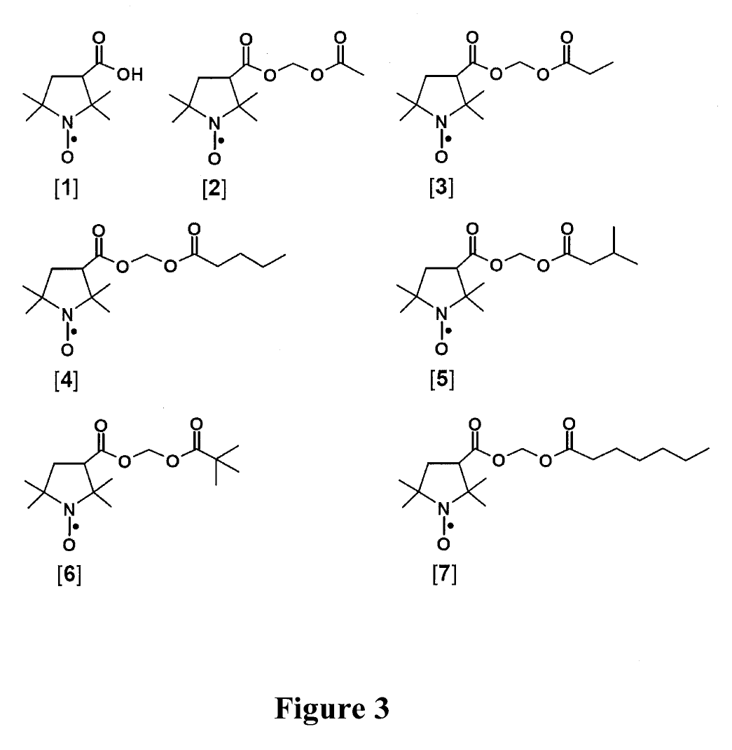

Reagents and solvents from commercial vendors were used without further purification. Reagents were obtained from Aldrich Chemical Company (Milwaukee, Wis.), and solvents were from VWR (West Chester, Pa.). Silica gel (230-400 mesh) and TLC plates (Silica Gel 60 F254) were from EMD Chemicals Inc. (Gibbstown, N.J.). Cell culture media and biochemicals were from Invitrogen Corp. (Carlsbad, Calif.). FIG. 3 provides structures synthesized in the following section.

3-Carboxy-2,2,5,5-tetramethyl-1-pyrrolidinyloxyl [1] was prepared as described by Rozantsev (7). 3-Acetoxymethoxycarbonyl-2,2,5,5-tetramethyl-1-pyffolidinyloxyl [2] was prepared as previously described (6). IR spectra were recorded on an FT-IR spectrometer (model 1600, Perkin-Elmer, Norwalk, Conn.) in CHCl3. Mass spectrometric analysis was performed by the Mass Spectrometry Facility in the Department of Chemistry and Biochemistry at the University of Maryland, College Park. Elemental analyses were performed by Atlantic Microlab, Inc. (Norcross, Ga.). Origin 8.0 software (OriginLab Corp., Northampton, Mass.) was used for data analysis.

### Synthesis of 3-(2,2-Dimethypropanoyl)oxycarbonyl-2,2,5,5-tetramethyl-1-pyrrolidinyloxyl [6].

To a solution of nitroxide [1] (0.25 g, 1.3 mmol) in DMSO (2 mL) was added K₂CO₃ (0.37 g, 2.7 mmol). After the reaction mixture was stirred at room temperature for 5 min, chloromethyl pivalate (0.2 g, 0.19 mL, 1.4 mmol) was added. Stirring was continued at room temperature for 3 h before the mixture was diluted with CH₂Cl₂ (50 mL) and brine (100 mL). The organic layer was dried over anhydrous MgSO₄ and evaporated under high vacuum to remove traces of DMSO. The resulting crude product was purified by chromatography on silica gel; elution with 1% (v/v) acetone in CHCl₃ afforded compound [6] (0.29 g, 75% yield). IR (CHCl₃): 1753 cm⁻¹ (broad ester peak). Anal. calculated for C₁₅H₂₆NO₅, C, 59.98; H, 8.73; N, 4.66; found, C, 59.90; H, 8.84; N, 4.53.

### Synthesis of 3-Chloromethoxycarbonyl-2,2,5,5-tetramethyl-1-pyrrolidinyloxyl [8].

This compound was prepared following the general procedure of Harada et al. (8). A mixture of CH₂Cl₂ (20 mL) and 20 mL of aqueous solution containing 3-carboxy-2,2,5,5-tetramethyl-1- pyrrolidinyloxyl [1] (0.5 g, 2.7 mmol), NaHCO₃ (0.9 g, 10.8 mmol), and tetrabutylammonium bisulfate (91 mg, 0.27 mmol) was stirred for 10 min at room temperature before addition of chloromethyl chlorosulfate (0.54 g, 0.33 mL, 3.3 mmol). The reaction mixture was vigorously stirred at room temperature for 2 h, during which the yellow color of the nitroxide moved from the aqueous phase into the organic phase. The CH₂Cl₂ phase was separated, washed with brine (100 mL), dried over anhydrous Na₂SO₄, filtered, and evaporated under reduced pressure to yield compound [8] (0.54 g, 85% yield). TLC (silica gel, 2:1 (v/v) hexane/EtOAc) showed only one spot. The product was used in subsequent reactions without further purification.

### Synthesis of 3-pentanoylmethoxycarbonyl-2,2,5,5-tetramethyl-1-pyrrolidinyloxyl [4].

A mixture of chloromethyl ester [8] (0.75 g, 3.2 mmol), K₂CO₃ (0.88 g, 6.4 mmol) and DMSO (2 mL) was stirred at room temperature for 5 min before addition of pentanoic acid (0.33 g, 0.34 mL, 3.2 mmol) and a few crystals of Nal. The reaction mixture was stirred at room temperature overnight and then diluted with CH₂Cl₂ (50 mL) and brine (100 mL). The organic phase was dried over anhydrous MgSO₄, filtered, and evaporated under high vacuum to remove traces of DMSO. The resulting crude product was purified on silica gel (7:3 (v/v) hexane:EtOAc) to give compound [4] (0.76 g, 80% yield). IR (CHCl₃): 1753 cm⁻¹ (broad ester peak)). Anal. calculated for C₁₅H₂₆NO₅: C, 59.98; H, 8.73; N, 4.66; found, C, 59.94; H, 8.83; N, 4.72.

### Synthesis of 3-propanoylmethyloxycarbonyl-2,2,5,5-tetramethyl-1-pyrrolidinyloxyl [3].

This nitroxide was synthesized using propionic acid in the procedure for nitroxide [4]; the yield was 85%. IR (CHCl₃): 1752 cm⁻¹ (broad ester peak). HR FAB MS (m/z): calculated for C₁₂H₂₂NO₅ (M⁺) 272.1498; found, 272.1498.

### Synthesis of 3-(3-methylbutanoyl)methoxycarbonyl-2,2,5,5-tetramethyl-1-pyrrolidinyloxyl [5].

This nitroxide was synthesized using isovaleric acid in the procedure for nitroxide [4]; the yield was 74%. IR (CHCl₃): 1753 cm⁻¹ (broad ester peak). Anal. calculated for C₁₅H₂₆NO₅: C, 59.98; H, 8.73; N, 4.66; found: C, 59.86; H, 8.81; N, 4.71.

### Synthesis of 3-heptanoylmethoxycarbonyl-2,2,5,5-tetramethyl-1-pyrrolidinyloxyl [7].

This nitroxide was synthesized using heptanoic acid in the procedure for nitroxide [4]; the yield was 84%. IR (CHCl₃): 1753 cm⁻¹ (broad ester peak). Anal. calculated for C₁₅H₂₆NO₅: C, 62.17; H, 9.21; N, 4.26; found: C, 62.33; H, 9.40; N, 4.22.

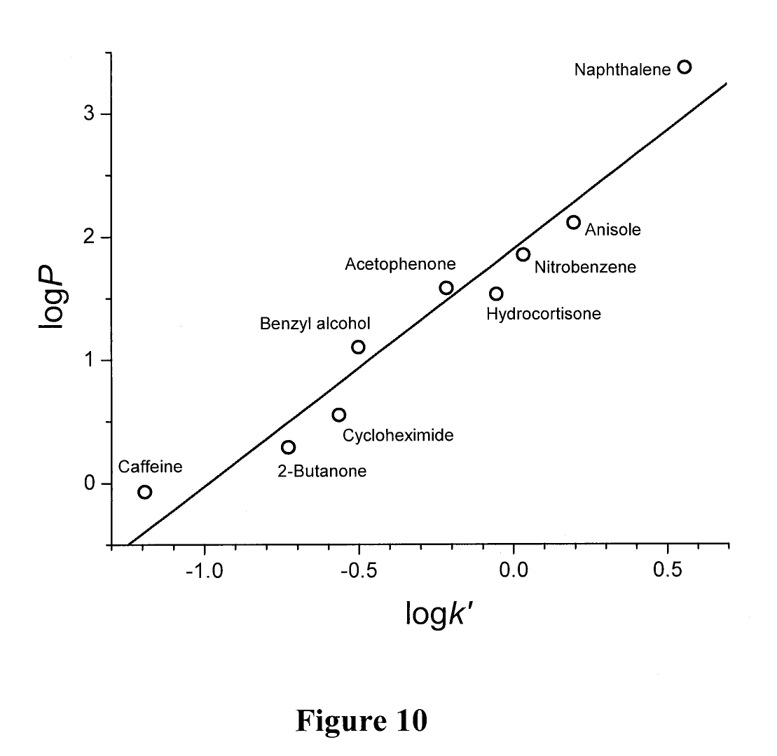

Determination of the logP values of labile esters. For each of the labile esters, the log10 of the octanol-water partition coefficient (logP) was determined. The logP values of ester 2-7 were evaluated from their capacity factors (k′) as determined by HPLC on a reverse-phase column (Kromasil 100 RP-C8; Higgins Analytical, Mountain View, Calif.), with a mobile phase consisting of methanol and 0.02 M sodium phosphate buffer (pH 6.0) in a volume ratio of 7:3 (9,10). The measurements were performed using isocratic elution (1 mL/min) on a Waters HPLC equipped with a diode-array detector (Model 600; Milford, Mass.); the absorbance at 222 nm was used to construct the chromatograms. The capacity factor is defined as k′=(tR−t0)/t0, where tR and t0 are, respectively, the elution times of the compound of interest and the void marker (thiourea). To generate a logP vs. logk′ calibration line, we used 9 compounds with known logP values (in parentheses): caffeine (−0.07), 2-butanone (0.29), cycloheximide (0.55), benzyl alcohol (1.10), hydrocortisone (1.53), acetophenone (1.58), nitrobenzene (1.85), anisole (2.11) and naphthalene (3.37) (11,12). The calibration line, as shown in FIG. 10, was used to convert the logk′ values of esters 2-7 into the corresponding logP values. Two to four replicates of each measurement were made.

Cellular Loading of Nitroxides. Jurkat lymphocytes were cultured and loaded with nitroxides as previously described (5,6). Briefly, a suspension of 1.8×10⁷ cells, at a density of 2×10⁷ mL⁻¹, were incubated for 70 min at room temperature in serum-free RPMI 1640 medium containing the indicated concentration of the appropriate nitroxide labile ester and 0.0015% (w/v) of the surfactant Pluronic F-127 (BASF Corp, Florham Park, N.J.). After incubation, cells were washed 3 times in Hanks' Balanced Salt Solution (HBSS), and resuspended in 400 mL HBSS. To lyse cells, 120 mM digitonin was added to each cell suspension, which was then sonicated for 1 mM in a bath sonicator (model G1 12SPIG, Laboratory Supplies Company, Inc., Hicksville, N.Y.). The lysate was assayed for nitroxide content by EPR spectroscopy. Each loading experiment was performed in triplicate for compounds [2], [3], [4], and [7], and once for compounds [5] and [6].

EPR Spectroscopy. EPR spectra were recorded on an X-band spectrometer (model E-109, Varian Inc., Palo Alto, Calif.) at the following settings: microwave power, 20 mW; microwave frequency, 9.55 GHz; field set, 3335 G; modulation frequency, 1 kHz; modulation amplitude, 0.5 G; field sweep, 80 G at 26.7 G min⁻¹ Where the amplitude of only the center spectral line was required, the sweep width was 8 G. EWWIN software (Scientific Software Solutions, Northville, Mich.) was used for spectrometer control and data acquisition. Nitroxide signal was measured as the peak-to-trough amplitude of the center line of the three-line spectrum.

### Discussion

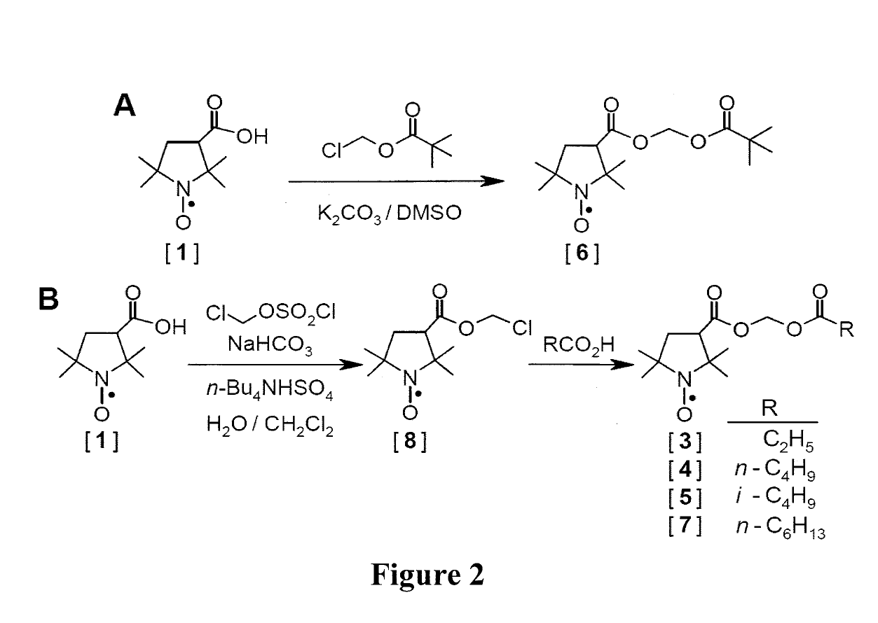

To adjust the lipophilicity of EPR pro-imaging agents systematically, a series of alkanoyloxymethyl esters of [1] were synthesized in which the hydrophobicity and steric bulk of the alkanoyl moiety was varied, as shown in FIG. 2. Compound [6] was synthesized through the procedure developed for [2] (6), with commercially available chloromethyl pivalate used instead of bromomethyl acetate as the alkylating agent to esterify nitroxide [1] as shown in FIG. 2A. However, for compounds [3], [4], [5] and [7], it was simpler to prepare the chloromethyl ester [8], which could undergo reaction with a series of inexpensive carboxylic acids to yield the corresponding labile esters, as shown in FIG. 2B.

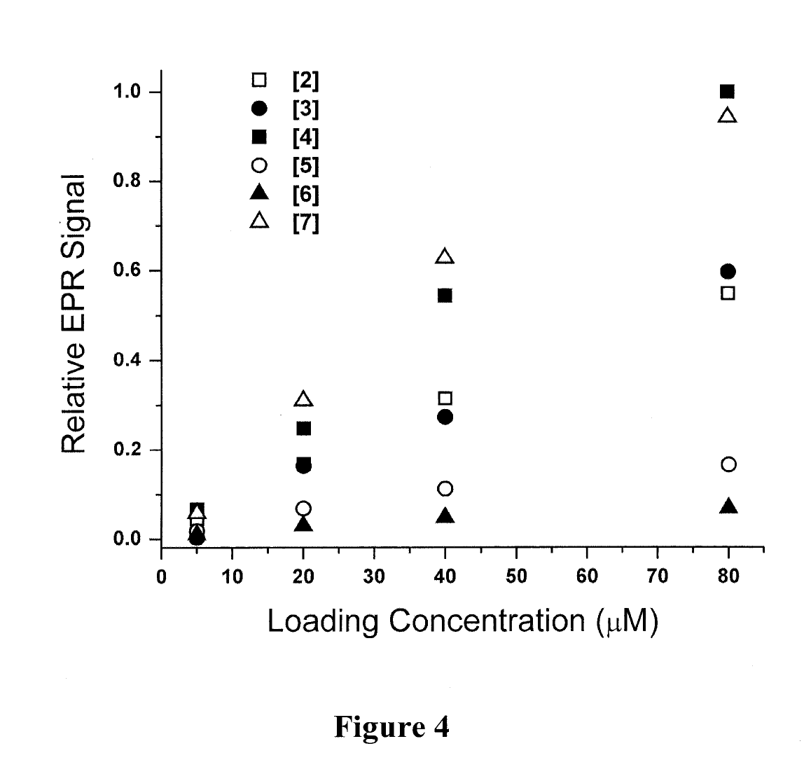

To investigate the effectiveness of the various labile esters to deliver nitroxide [1] intracellularly, Jurkat lymphocytes were incubated with the esters over a range of concentrations and assayed the intracellular content of nitroxide [1]. Initially, the intracellular loading of the esters were compared after incubation with esters [2], [3], [4], and [7]—a family in which the length of the alkanoyl chain was systematically increased. The results are presented in FIG. 4. It can be seen that while increasing the alkanoyl chain length from 2 to 3 had negligible effect on intracellular loading (compare results for esters [2] and [3]), increasing the chain length to 5 (ester [4]) resulted in a substantial enhancement of intracellular loading. A further increase of the chain length to 7 (ester [7]), however, brought no additional improvement in loading, beyond that of 5.

The effect of branching in the alkanoyl chain in the labile ester was investigated on intracellular loading of nitroxide [1] by incubating the cells with the isomeric esters [4], [5], and [6]. The results in FIG. 4 show that while ester [4], with an n-pentanoyl moiety, gave high intracellular levels of nitroxide [1], esters [5] and [6], with 3-methylbutanoyl (isovaleryl) and 2,2-dimethylpropanoyl (pivaloyl) moieties, respectively, gave very poor intracellular loading of nitroxide [1]. These findings indicate that increased branching in the alkanoyl chain drastically diminished the ability of the labile ester to deliver nitroxide [1] intracellularly.

Besides changing the steric bulk of the labile ester, increased branching in the alkanoyl chain is also expected to decrease the lipophilicity of the molecule. A commonly-used measure of lipophilicity is the log₁₀ of the octanol-water partition coefficient, logP. The experimentally determined logP values for esters 2-7 (Table 1) show that lipophilicity does decrease with increased alkanoyl chain branching, but the effect is very slight.

TABLE I
LogP values of nitroxide labile esters [2]-[7]

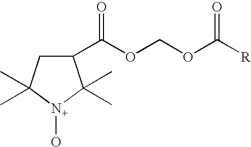

| Ester | R | logP |
|---|---|---|
| 2 | —CH₃ | 1.12 |
| 3 | —CH₂CH₃ | 1.33 |
| 4 | —(CH2)₃CH₃ | 2.67 |
| 5 | —CH₂CH(CH₃)₂ | 2.62 |
| 6 | —C(CH₃)₃ | 2.60 |
| 7 | —(CH₂)₅CH₃ | 3.54 |

Thus, the isomeric esters [4], [5] and [6], with primary, secondary and tertiary alkanoyl chains, respectively, have logP values that decrease systematically but slightly, from 2.67 down to 2.60. Moreover, intracellular loading of the branched-chain esters [5] and [6] is clearly much less effective than that of straight-chain esters that are either less lipophilic ([2] and [3]) or more lipophilic ([7]). Therefore, the differences in lipophilicity cannot be invoked to explain differential intracellular loading. A reasonable inference is that intracellular esterases that hydrolyze alkanoyloxymethyl esters have a strong preference for straight over branched alkanoyl chains.

Even though O₂ has two unpaired electrons, one on each oxygen atom, EPR spectroscopy cannot directly identify O₂ at 37° C.; instead the detection of which requires the interaction of a stable free radical with O₂. Nitroxides are considered stable free radicals whose ERR spectral lines are broadened in the presence of O₂ due to the interaction of the two paramagnetic species. FIG. 6 shows the linearity of nitroxide [1] EPR spectral linewidth as O₂ concentration increases from 0% to 21%.

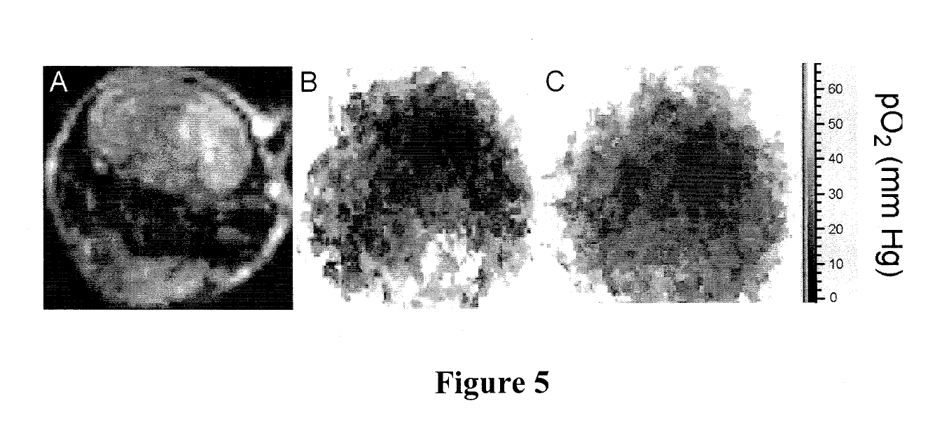

In vivo EPR mapping of the changes of pO2 in the brain of a mouse induced by ischemic stroke is shown in FIG. 5. After the mouse was given nitroxide [1] via ip injection, 3-D spectral-spatial images of nitroxide in the brain were obtained. After converting the nitroxide EPR spectral linewidth into pO₂ values, tissue pO₂ distribution before (panel C) and after (panel B) middle cerebral artery occlusion (MCAO), an animal stroke model, was mapped. The spatial resolution is approximately 0.3 mm and pO₂ resolution about 5 mmHg The results showed that the hypoxic region of the pO₂ image match well with the infarction area of the MR diffusion image (panel A). It is important to recognize that the results shown in FIG. 5 show that nitroxide [1] after ip injection can quantitate O₂ levels in the brain. After a mouse was given nitroxide [1] and prior to inducing the stroke, pO₂ levels were normal (panel C). Thereafter, a marked decrease in pO₂ was noted after stroke was induced in which O₂ delivery into the occluded region was prevented (panel B). After the stroke, the region of the brain affected continued to use O₂ in an attempt to maintain normal brain function, e.g., biosynthesis and metabolism of neurotransmitters, despite the lack of O₂ influx into this region. At the 30 min time point, the significant decrease in O₂ levels (panel B) must undoubtedly have had profound effects on brain function.

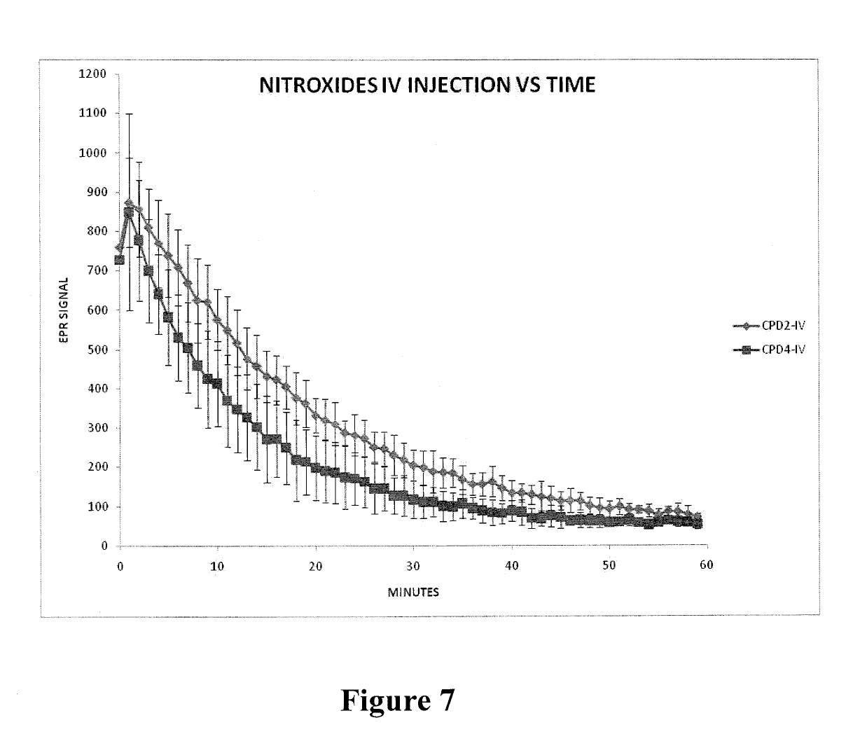

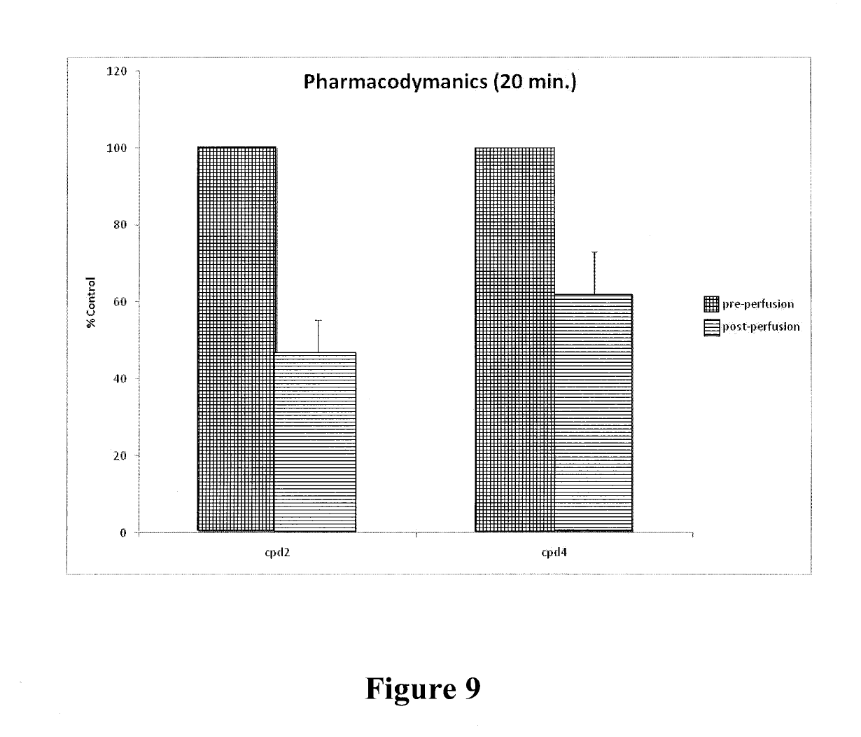

Further in vivo data is shown in FIGS. 7-9 wherein unexpected and surprising it was found that nitroxide [4] far surpasses the results shown in nitroxide [2]. 3-Acetoxymethoxycarbonyl-2,2,5,5-tetramethyl-1-pyrrolidinyloxyl [2] is CPD2 in FIG. 7 whereas 3-pentanoylmethoxycarbonyl-2,2,5,5-tetramethyl-1-pyrrolidinyloxyl [4] is CPD4. Each of the nitroxides was given iv into mice. FIG. 7 is graph showing the amount of the concentration of the nitroxide in the head of a mouse; in the vasculature as well as in the brain. Clearly it is evident that the [4] molecule is reduced because it has entered into the brain pass the BBB and been converted to nitroxide [1] by the esterases therein.

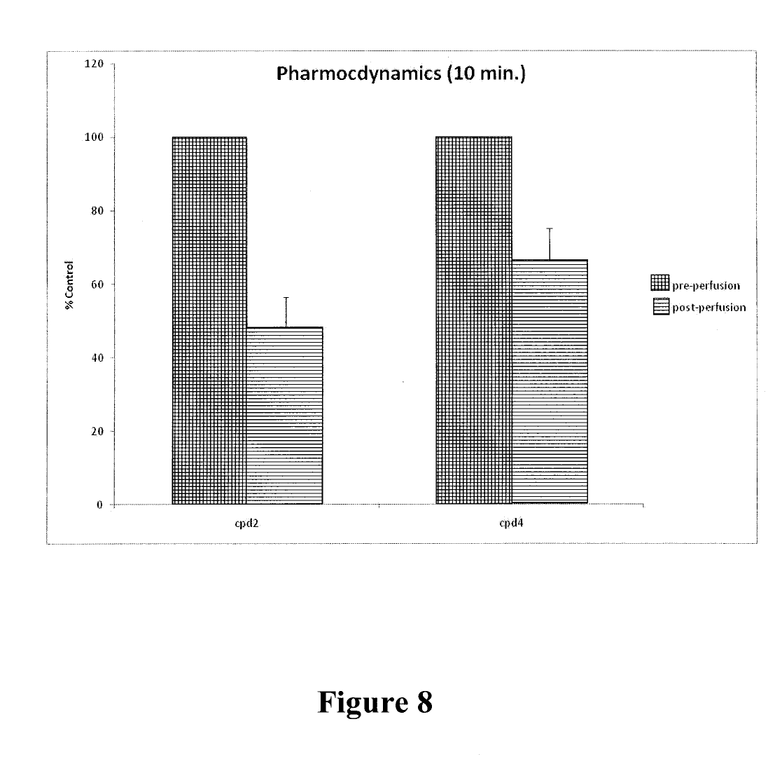

At the 10-min point in the time period depicted in FIG. 7, the mouse was removed from the EPR imager, and its vasculature leading to the head was washed with normal saline. The mouse was returned to the EPR imager and again EPR spectra were recorded as shown in FIG. 8. These data show that about 42% of the original dose of nitroxide [2] had crossed the blood brain barrier and after hydrolysis to 3-carboxy-2,2,5,5-tetramethyl-1-pyrrolidinyloxyl [1], this nitroxide was entrapped in the brain. However, in the case of 3-pentanoylmethoxycarbonyl-2,2,5,5-tetramethyl-1-pyrrolidinyloxyl [4], data show that about 65% of the original dose of nitroxide [4] had crossed the blood brain barrier and after hydrolysis to 3-carboxy-2,2,5,5-tetramethyl-1-pyrrolidinyloxyl [1], this nitroxide was entrapped in the brain.

At the 20-min point in the time period as shown in FIG. 7, the mouse was removed from the EPR imager, and its vasculature leading to he head was washed with normal saline. The mouse returned to the EPR imager and again EPR spectra were recorded. These data show that about 42% of the original dose of nitroxide [2] had crossed the blood brain barrier and after hydrolysis to 3-carboxy-2,2,5,5-tetramethyl-1-pyrrolidinyloxyl [1], this nitroxide was entrapped in the brain. In the case of 3-pentanoylmethoxycarbonyl-2,2,5,5-tetramethyl-1-pyrrolidinyloxyl [4], data show that about 65% of the original dose of nitroxide [4] had crossed the blood brain barrier and after hydrolysis to 3-carboxy-2,2,5,5-tetramethyl-1-pyrrolidinyloxyl [1], this nitroxide was entrapped in the brain. Of course the concentration of each nitroxide in the mouse head is lower at 20 min than at 10 min, but the percentage in the brain is unaltered.

### REFERENCES

The contents of the references cited herein are incorporated by reference herein for all purposes.

1. Baudelet, C., and Gallez, B. (2002) How does blood oxygen level-dependent (BOLD) contrast correlate with oxygen partial pressure (pO2) insider tumors. Magn. Reson. Med. 48, 980-986.

2. Elas, M., Williams, B. B., Parasca, A., Mailer, C., Pelizzari, C. A., Lewis, M. A., River, J. N., Karczmar, C. S., Barth, E. D., and Halpern, H. J. (2003) Quantitative tumor oxymetric images from 4D electron paramagnetic resonance imaging (EPRI): Methodology and comparison with blood oxygen level-dependent (BOLD) MRI. Magn. Reson. Med. 49, 682-691.

3. Matsumoto, K., Bernardo, M., Subramanian, S., Choyke, P., Mitchell, J. M., Krishna, M. C., and Lizak, M. J. (2006) MR assessment of changes of tumor in response to hyperbaric oxygen treatment. Magn. Reson. Med. 56, 240-246.

4. Shen, J., Liu, S., Miyake, M., Liu, W., Pritchard, A., Kao, J. P. Y., Rosen, G. M., Tong, Y., and Liu, K. J. Use of 3-acetoxymethoxycarbonyl-2,2,5,5-tetramethyl-1-pyrrolidinyloxyl as an EPR oximetry probe: potential for in vivo measurement of tissue oxygenation in mouse brain. Magn. Reson. Med. 55, 143 3-1440.

5. Rosen, G. M., Burks, S. R., Kohr, M. J., and Kao, J. P. Y. (2005) Synthesis and biological testing of aminoxyls designed for long-term retention by living cells. Org. Biomolec. Chem. 3, 645-648.

6. Kao, J. P. Y., and Rosen, G. M. (2004) Esterase-assisted accumulation of 3-carboxy-2,2,5,5-tetramethyl-1-pyrrolidinloxyl into lymphocytes. Org. Biomol. Chem. 2, 99-102.

7. Rozantsev, E. G. (1970) Free Nitroxyl Radicals. pp 206, Plenum Press, New York.

8. IIarade, N., Hongu, M., Tanaka, T., Kawaguchi, T., Hashiyama, T., and Tsujihara, K. (1994) A simple preparation of chloromethyl esters of the blocked amino acids. Syn. Comm. 24, 767-772.

9. Lambert, W. J. (1993) Modeling oil-water partitioning and membrane permeation using reverse-phase chromatography. J. Chromatogr. A. 656, 469-484.

10. Namjesnik-Dejanovic, K., and Cabaniss, S. E. (2004) Reverse-phase HPLC method for measuring polarity distributions of natural organic matter. Environ. Sci. Technol. 38, 1108-1114.

11. Leo, A. IIansch, C., and Elkins, D. (1971) Partition coefficients and their uses. Chem. Rev. 71, 525-616.

12. Singh, P., and Roberts, M. S. (1996) Local deep tissue penetration of compounds after transdermal application: Structure-tissue penetration relationships. J. Pharmacol. Exp. Ther. 279, 908-917.

13. Pou S, Davis P L, Wolf G L, Rosen G M. Use of nitroxides as NMR contrast enhancing agents for joints. Free Radi.c Res. 1995; 23:353-364.

1. A method of determining concentration levels of O₂ in tissue of a subject in need of such determination, the method comprising:
administering to the subject a nitroxide compound having the structure 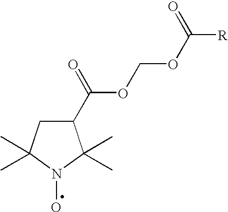
wherein R is a straight-chain alkyl group of the general formula (CH₂)ₙCH₃ wherein n can be 1 to 15, in an amount sufficient to accumulate in such tissue; and
quantitating the concentration of O₂ in the tissue.

2. The method according to claim 1, wherein the tissue is brain tissue.

3. The method according to claim 2, wherein the nitroxide crosses the blood-brain barrier.

4. The method according to claim 1, wherein the concentration of O₂ in the tissue is measured by electron paramagnetic resonance (EPR) spectroscopy.

5. The method according to claim 1, wherein the concentration of O₂ in the tissue is measured by (EPR) spectroscopy and wherein the EPR spectral linewidth of the administered compounds increases relative to the increase concentration of O₂.

6. The method according to claim 1, wherein n is 2-5.

7. The method according to claim 1, wherein the tissue is tumorous, dermis, hypodermis, and/or muscle.

8. A method of determining concentration levels of O₂ in brain tissue in need of such determination, the method comprising:
administering to the brain tissue a 3-alkanoyloxymethoxycarbonyl nitroxide ester having the structure 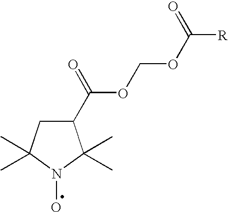
wherein R is a straight-chain alkyl group having the general formula (CH₂)ₙCH₃ wherein n can be 1 to 15, in an amount sufficient to cross the blood-brain barrier and accumulates in brain tissue for hydrolysis therein thereby generating 3-carboxy-2,2,5,5-tetramethyl-1-pyrrolidinyloxyl having the structure: 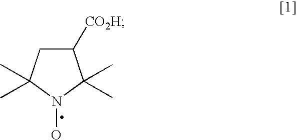
and
determining the amount of accumulated 3-carboxy-2,2,5,5-tetramethyl-1-pyrrolidinyloxyl and/or any remaining 3-alkanoyloxymethoxycarbonyl nitroxide ester to determine the levels of O₂ is such tissue.

9. The method according to claim 8, wherein the nitroxide crosses the blood-brain barrier.

10. The method according to claim 8, wherein the concentration of O₂ in the tissue is measured by electron paramagnetic resonance (EPR) spectroscopy.

11. The method according to claim 8, wherein the concentration of O₂ in the tissue is measured by (EPR) spectroscopy and wherein the EPR spectral linewidth of the administered compounds increases relative to the increase concentration of O₂.

12. The method according to claim 8, wherein n is 2-5.

13. The method according to claim 8, wherein the nitroxide compound is

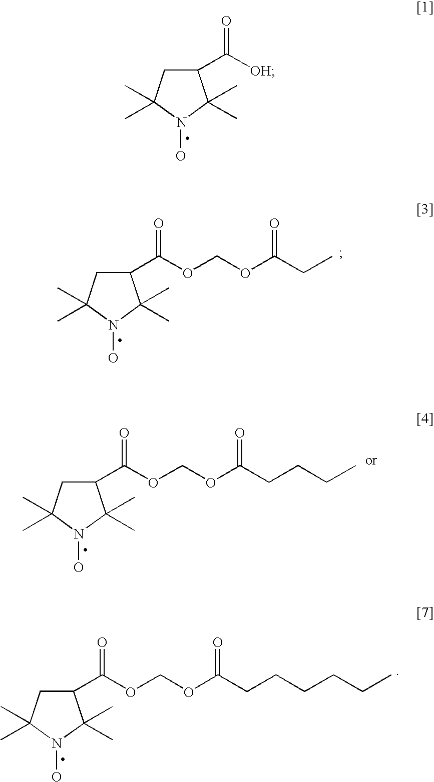

14. A method for providing a EPR oximetry probe to map O₂ levels in testing tissue, the method comprising:
administering to the tissue a nitroxide compound having the structure 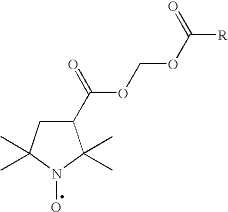
wherein R is a straight-chain alkyl group having the general formula (CH₂)ₙCH₃ wherein n can be 1 to 15.

15. The method according to claim 14, wherein the nitroxide compound is

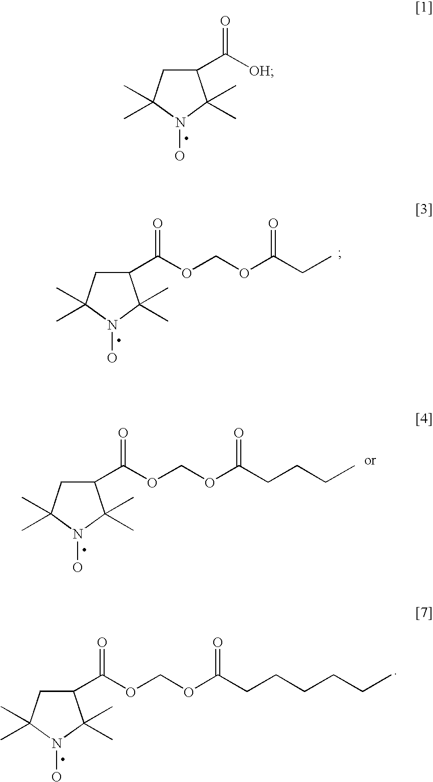

16. The method according to claim 14, wherein the nitroxide crosses the blood-brain barrier.

17. The method according to claim 14, wherein the EPR spectral linewidth of the administered nitroxide compound increases relative to the increase concentration of O₂.

18. A method to evaluate oxygen concentration in a biological system, comprising the steps of:
(1) introducing physiologically acceptable paramagnetic material to the biological system;
(2) applying an electromagnetic field to the biological system; and
(3) determining the EPR spectra of the biological system, wherein the paramagnetic material is a compound a compound having a structure 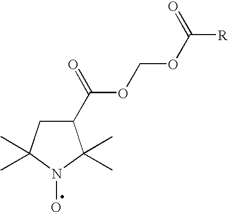
wherein R is a straight-chain alkyl group having the general formula (CH₂)ₙCH₃ wherein n can be 1 to 15.

19.-20. (canceled)
